(19) United States
(12) Patent Application Publication
(10) Pub. No.: US 2023/0074858 A1
(43) Pub. Date: Mar. 9, 2023

(54) METHOD FOR TRACKING PUPIL FOR EYE IN VARIOUS CONDITIONS, AND HEALTH DIAGNOSIS SYSTEM USING SAME

(71) Applicant: GoQba Technology Corp., Gyeonggi-do (KR)

(72) Inventors: Jin Young Park, Seoul (KR); Jeevan Kharel, Seoul (KR)

(21) Appl. No.: 17/780,097

(22) PCT Filed: Oct. 13, 2020
(86) PCT No.: PCT/KR2020/013909

§ 371 Date: Oct. 12, 2022

(30) Foreign Application Priority Data
Nov. 27, 2019 (KR) 10-2019-0154823

(51) Int. Cl. A61B 3/113; G06V 40/18; G06T 7/70; G06T 7/00; G06T 7/20; A61B 3/00

(52) U.S. Cl. CPC A61B 3/113; A61B 3/0033; A61B 3/0041; G06T 7/0012; G06T 7/20; G06T 7/70; G06V 40/18; G06T 2207/20084; G06T 2207/30041; G06V 2201/03

(57) ABSTRACT

A method for tracking a pupil according to an embodiment of the present invention includes: detecting an upper eyelid and a lower eyelid in eyes; detecting an opening degree of the eye; detecting the pupil between the upper eyelid and the lower eyelid; generating a pupil coordinate in accordance with the detected pupil; and calculating a coordinate value for one point of the pupil in the pupil coordinate.

### TECHNICAL FIELD

The present invention relates to a method for tracking a pupil and a medical examination system using the same, and more particularly, to a method for tracking a pupil, which can track pupils of persons having various states such as different eye sizes and makeup, and enables medical examination through movements of the tracked pupils, and a medical examination system using the same.

### BACKGROUND ART

Dizziness is a common symptom of many people, and can cause serious problems in everyday life by being chronic or a reucurrent attack. Causes of the dizziness can be divided into four categories, i.e., an ear problem, a brain problem, an internal medicine problem, and a psychological problem. The people can do their daily life without any inconvenience because the vestibular organ in the ear maintains the balance of the body, but if an ear problem occurs, then dizziness occurs due to abnormality in the vestibular organ. Dizziness is a common side effect in addition to the onset of loss of language disorders, cognitive and/or motor skills. Further, dizziness with arrhythmia, hypoglycemia, oriental hypertension, vagal syncope, etc., may be caused by the internal medicine problem, and dizziness with panic disorder, agoraphobia, anxiety disorder, depression, etc., may be caused by the psychological problem.

On the other hand, dizziness caused by the brain problem is associated with brain diseases such as Alzheimer’s disease, which causes dementia, and can be confirmed through the movement of the eyeball. In order to confirm the movement of these eyeballs, a method of tracking the pupil is generally done. Since equipment for tracking the pupil is generally expensive, it is a burdensome situation to install and use in medical institutions, except for large medical institutions. Further, the equipment for tracking the pupil is expensive, but most equipment is designed based on persons having large eyes and it is difficult to track the pupil of a person having small eyes, and a portion where makeup around the eyes is made and the pupil cannot be distinguished, so it is also difficult to track the pupil of a person who wears makeup. Here, the makeup around the eye refers to various beauty procedures and surgery, including eyelines, eyelashes, tattoos, etc., applied to a portion around the eyes. As a result, people have a hassle of opening their eyes and removing makeup for pupil tracking.

### DISCLOSURE

### Technical Problem

A technical problem to be solved by a method for tracking a pupil and a medical examination system using the same according to the technical spirit of the present invention is to provide a method for tracking a pupil, which enables pupil tracking for an eye in which makeup is applied and a part of the pupil is covered, and a medical examination system using the same.

Further, a technical problem to be solved by a method for tracking a pupil and a medical examination system using the same according to the technical spirit of the present invention is to provide a method for tracking a pupil, which enables remotely checking a health state through pupil tracking, and a medical examination system using the same.

In addition, a technical problem to be solved by a method for tracking a pupil and a medical examination system using the same according to the technical spirit of the present invention is to provide a method for tracking a pupil, which enables checking a health state at low cost, and a medical examination system using the same.

A technical problem to be solved by a method for tracking a pupil and a medical examination system using the same according to the technical spirit of the present invention is not limited to the problem mentioned above, and another problem not mentioned will be clearly appreciated by those skilled in the art from the following disclosure.

### Technical Solution

By a technical idea of the present invention, a method for tracking a pupil according to an embodiment includes: detecting an upper eyelid and a lower eyelid in eyes; detecting an opening degree of the eye; detecting the pupil between the upper eyelid and the lower eyelid; generating a pupil coordinate in accordance with the detected pupil; and calculating a coordinate value for one point of the pupil in the pupil coordinate.

Further, the detecting of the upper eyelid and the lower eyelid in the eyes includes detecting a makeup portion between the upper eyelid and the lower eyelid, and is achieved by a convolutional neural network.

In addition, in the detecting of the pupil between the upper eyelid and the lower eyelid, in the opening degree of the eye, the pupil is detected between the upper eyelid and the lower eyelid while the upper eyelid and the lower eyelid are spaced apart from each other so that the pupil has a visibility.

Furthermore, in the detecting of the pupil between the upper eyelid and the lower eyelid, pupil tracking technologies are differently applied according to the opening degree of the eye.

By a technical idea of the present invention, a medical examination system according to an embodiment includes: a detection unit detecting an eye of a user, detecting an opening degree of the eye and a pupil through the opened eye, and generating an image of the detected eye; a control unit connected to the detection unit, and receiving an image signal according to the image from the detection unit and storing the image according to the image signal; and an expert terminal connected to the control unit to receive the image signal according to the image from the control unit, and output the image according to the image signal and receive a comment, and the detection unit detects an upper eyelid and a lower eyelid in the eye, detects the opening degree of the eye, and detects the pupil between the upper eyelid and the lower eyelid, and the expert terminal receives a comment through an expert interface and transmits a comment signal according to the comment to the control unit.

Further, the detecting unit includes an opening detection unit detecting the eye of the user, detecting the upper eyelid and the lower eyelid of the eye, and detecting the opening degree of the eye, a pupil detection unit detecting the pupil between the upper eyelid and the lower eyelid detected by the opening detection unit, an operating unit generating a pupil coordinate according to the pupil detected by the pupil detection unit, and calculating a coordinate according to one point of the pupil, and a processing unit connected to the opening detection unit, the pupil detection unit, and the operating unit to generate images for the upper eyelid and the lower eyelid detected by the opening detection unit, and the pupil detected by the pupil detection unit, include a coordinate value of one point of the pupil calculated by the operating unit be in the image, and transmit and receiving a signal.

Further, the processing unit generates the image signal according to the image and transmits the image signal to the control unit, the control unit receives an image signal according to an image including a movement of the pupil corresponding to a case where the user is in an abnormal health state and transmits the image signal to the expert terminal, and the expert terminal outputs the image according to the transmitted image signal, and receives a comment according to the output image, and generates the comment signal according to the comment and transmits the generated comment signal to the control unit.

Further, the medical examination system further includes a guardian terminal connected to the control unit to receive the signal from the control unit, and the control unit receives an image signal according to the image including the movement of the pupil corresponding to the case where the user is in the abnormal health state and transmits the image signal to the guardian terminal, and transmits the comment signal according to the comment transmitted from the expert terminal to the guardian terminal, and the guardian terminal outputs the image according to the transmitted image signal and the comment according to the comment signal.

Further, the detection unit further includes a control command output unit capable of outputting a command voice and a command screen, and the control command output unit outputs the command voice and the command screen to induce the movement of the pupil of the user.

Further, the opening detection unit detects a makeup portion between the upper eyelid and the lower eyelid.

Further, when the opening detection unit detects the opening degree of the eye in which the upper eyelid and the lower eyelid are spaced apart from each other so that the pupil has a visibility, the pupil detection unit detects the pupil between the upper eyelid and the lower lid.

Further, the expert terminal receives a command of the movement of the pupil through the expert interface and transmits the command signal of the movement of the pupil to the control unit.

The detection unit receives the command signal of the movement of the pupil from the control unit, and outputs the command signal to induce the movement of the pupil of the user.

Further, the expert terminal includes the expert interface for receiving the command of the movement of the pupil, and the expert interface includes a screen mark movement layer capable of moving a mark by an expert, a voice input layer through which the expert is capable of inputting a voice, and a specific behavior induction layer through which the expert is capable of displaying an object for inducing a specific behavior.

### Advantageous Effects

A method for tracking a pupil and a medical examination system using the same according to embodiments by the technical spirit of the present invention have the following effects.

(1) Pupil tracking for the eye in which the makeup is applied and a part of the pupil is covered can be made.

(2) The health state can be checked even remotely through the pupil tracking.

(3) The health state can be checked even at low cost.

However, effects which can be achieved by a method for tracking a pupil and a medical examination system using the same according to an embodiment of the present invention are not limited to the effects mentioned above, and other effects not mentioned will be clearly appreciated by those skilled in the art from the following disclosure.

### DESCRIPTION OF DRAWINGS

A brief description of each drawing is provided for more sufficient understanding of the drawings cited in the present specification.

FIG. 1 is a flowchart illustrating a method for tracking a pupil according to an embodiment of the present invention.

FIG. 2 is a diagram illustrating a medical examination system according to an embodiment of the present invention.

FIG. 3 illustrates an example of a detection unit of the medical examination system according to an embodiment of the present invention.

### BEST MODE

The present invention may have various modifications and various embodiments and specific embodiments will be illustrated in the drawings and described in detail through the detailed description. However, this does not limit the present invention to the specific embodiments, and it should be understood that the present invention covers all the modifications, equivalents and replacements included within the idea and technical scope of the present invention.

In describing the present invention, a detailed description of related known technologies will be omitted if it is determined that they unnecessarily make the gist of the present invention unclear. In addition, numeral figures (for example, first, second, and the like) used during describing the specification are just identification symbols for distinguishing one element from another element.

Further, in the present specification, if it is described that one component is “connected to” or “accesses” the other component, it is understood that the one component may be directly connected to or may directly access the other component but unless explicitly described to the contrary, another component may be “connected” or “access” between the components.

In addition, in the present specification, in the case of components expressed by ‘unit’, and the like, two or more components may be combined into one component or one component may be divided into two or more components for each more subdivided function. In addition, each of the components described below may additionally perform some or all of the functions that are handled by other components in addition to main functions that the corresponding component is responsible for, and some of the main functions of which the respective components are charge may be exclusively carried out by other components.

Hereinafter, embodiments according to the technical idea of the present invention will be described detail in order.

According to an embodiment of the present invention, a method for tracking a pupil may include: detecting an upper eyelid and a lower eyelid in eyes; detecting an opening degree of the eye; detecting the pupil between the upper eyelid and the lower eyelid; generating a pupil coordinate in accordance with the detected pupil; and calculating a coordinate value for one point of the pupil in the pupil coordinate.

Further, the detecting of the upper eyelid and the lower eyelid in the eyes may include detecting a makeup portion between the upper eyelid and the lower eyelid, and may be achieved by a convolutional neural network.

In addition, in the detecting of the pupil between the upper eyelid and the lower eyelid, in the opening degree of the eye, the pupil is detected between the upper eyelid and the lower eyelid while the upper eyelid and the lower eyelid are spaced apart from each other so that the pupil has a visibility.

Furthermore, in the detecting of the pupil between the upper eyelid and the lower eyelid, pupil tracking technologies may be differently applied according to the opening degree of the eye.

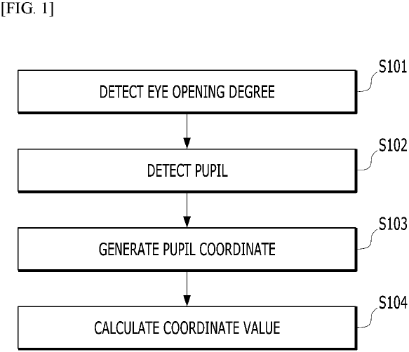

FIG. 1 is a flowchart illustrating a method for tracking a pupil according to an embodiment of the present invention.

The method for tracking a pupil according to an embodiment of the present invention illustrated in FIG. 1 may track a movement of a pupil by detecting the pupil in an eye which is in an opened state. Here, method for tracking a pupil according to the embodiment may detect the pupil for the opened eye and track the movement of the pupil regardless of an opened size of the eye.

First, a step (S101) of detecting an opening degree of the eye may be made. In step S101, upper eyelids and lower eyelids in the eyes may be detected. In step S101, each of the upper eyelids and the lower eyelids may be detected. While a lower end of the upper eyelids is connected to an upper end of the lower eyelids, the opening degree of the eye may be determined through a gap between the upper eyelids and the lower eyelids. Here, the upper eyelids and the lower eyelids may be detected in 3 dimensions through a convolutional neural network (CNN), and the opening degree of the eye may be detected. The CNN may be constituted by at least one convolutional layer, a pooling layer, and fully connected layers.

Actually, while the upper eyelid and the lower eyelid are spaced apart from each other, a part of the eyeball may be exposed between the upper eyelid and the lower eyelid. The opening degree of the eye may be divided into a closed state, a semi opened state, and a wide opened state. Here, the closed state means a state in which the pupil of the eyeball is fully covered by the upper eyelid and the lower eyelid and does not visibility, the semi opened state means a state in which the pupil of the eyeball is partially covered by the upper eyelid and the lower eyelid and is not exposed circularly, but has the visibility, and the wide opened state means a state in which the pupil of the eyeball is not covered by the upper eyelid and the lower eyelid and is circularly exposed and has the visibility.

Meanwhile, in the method for tracking the pupil, pupil tracking technology may be differently applied according to the opening degree of the eye. For example, in the closed state, steps to be described below may not be achieved and in the semi opened state and the wide opened state, the steps to be described below may be achieved. That is, the steps to be described below are preferably achieved while the gap between the upper eyelid and the lower eyelid is a set value or more so that the pupil of the eyeball may be exposed to have the visibility.

Further, in step S101, a step of detecting a makeup portion in the upper eyelid and the lower eyelid of the eye may be achieved. Here, the makeup portion means a portion when the makeup (e.g., the eyelines, the eyelashes, tattoos, etc.) is processed in the upper eyelid and the lower eyelid. The makeup portion may be detected through ResNet in the conventional neural network. Further, the makeup portion has a color, but may be distinguished from the pupil. As a result, since the makeup portion may not be detected by the pupil, the pupil may be more accurately detected.

Subsequently, a step (S102) of detecting the pupil may be achieved. In step S102, the pupil is detected while a gap between the upper eyelid and the lower eyelid detected through step S101 is detected. Here, in the eyeball between the upper eyelid and the lower eyelid, the pupil shows a black color unlike another portion of the eyeball showing a white color, and as a result, the pupil may be easily detected. As a result, even though the pupil is partially covered by the upper eyelid and the lower eyelid, the pupil may be detected.

Since the makeup portion is already detected in the upper eyelid and the lower eyelid through step S101, the pupil may be detected while being distinguished from the makeup portion.

Subsequently, a step (S103) of generating a pupil coordinate may be achieved. Step S103 may be achieved by using a regression tree ensemble based on the pupil detected through step S102. Here, the pupil coordinate may be achieved as a form of an XY coordinate or an XYZ coordinate by using one point (e.g., a center) of the detected pupil as a center. For example, an original point of the pupil coordinate corresponds to one point of the pupil, and one point of the pupil may be calculated as (0, 0) in the XY coordinate and calculated as (0, 0, 0) in the XYZ coordinate. Meanwhile, the pupil coordinate may be achieved by a coordinate considering the movement of the pupil by using the regression tree ensemble.

Subsequently, a step (S104) of calculating a coordinate value for one point of the pupil may be achieved. When the movement of the pupil is achieved, the coordinate value for one point of the pupil may be changed and calculated. That is, the changed coordinated value may be used for detecting the movement of the pupil.

The pupil tracking method according to the embodiment may detect makeup-processed portions of the upper eyelid and the lower eyelid of the eye in the semi opened state and the wide opened state of the eye, and detect the pupil by distinguishing from the makeup-processed portion. As a result, even when the pupil is partially covered by the eyelid and the makeup processing for the eye is achieved, the pupil is stably detected to achieve tracking according to the movement of the pupil. That is, in order to detect and track the pupil, a request for widely opening the eye or removing the makeup may not be made if the eye is not closed. Therefore, the pupil tracking method according to the embodiment easily detects the pupil of a person (e.g., Asian) having a relatively small eye to easily achieve the tracking of the pupil.

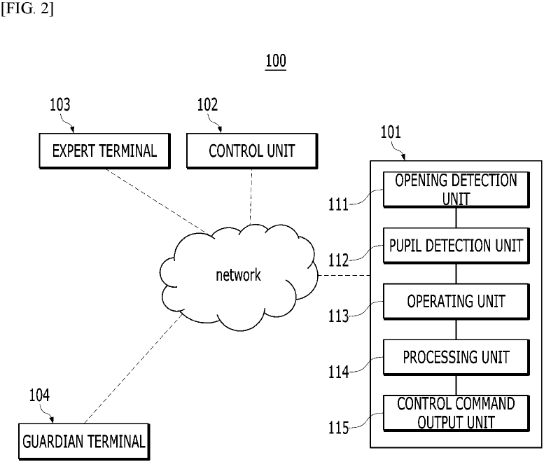

FIG. 2 is a diagram illustrating a medical examination system 100 according to an embodiment of the present invention.

As illustrated in FIG. 2, the medical examination system 100 according to an embodiment of the present invention may include a detection unit 101, a control unit 102, an expert terminal 103, and a guardian terminal 104, and diagnose the health state of the user by tracking the movement of the pupil of the user, and provide a medical service to the user. Here, the user may be persons of which continuous checking of the health state is required, e.g., the elderly, the disabled, patients, etc., and are active mainly in an indoor space.

Further, the detection unit 101, the control unit 102, the expert terminal 103, and the guardian terminal 104 may be connected to each other through a network. The network refers to a connection structure in which information may be exchanged between nodes such as a plurality of terminals and servers, and an example of such a network may include a 3rd Generation Partnership Project (3GPP) network, a Long Term Evolution (LTE) network, a 5G network, a World Interoperability for Microwave Access (WIMAX) network, Internet, a Local Area Network (LAN), Wireless Local Area Network (LAN), a Wide Area Network (WAN), a Personal Area Network (PAN), a Bluetooth network, a satellite broadcasting network, an analog broadcasting network, a Digital Multimedia Broadcasting (DMB) network, etc., but is not limited thereto.

The detection unit 101 is generally positioned in the indoor space, and detects the eye of the user to detect the opening degree of the eye and the pupil of the opened eye. Further, the detection unit 101 may generate the pupil coordinate for the detected pupil and calculate the coordinate value of one point of the pupil. The detection unit 101 may generate an image of the detected eye of the user, and the image may include the coordinate value of one point of the pupil. The detection unit 101 according to the embodiment may be configured in the form of a camera, and configured in the form of equipment including a camera lens, and positioned to correspond to the eye of the user.

Further, the detection unit 101 may include an opening detection unit 111, a pupil detection unit 112, an operating unit 113, and a processing unit 114.

The opening detection unit 111 detects the eye of the user, and may detect the opening degree of the eye. Here, the opening detection unit 111 detects the upper eyelid and the lower eyelid. While a lower end of the upper eyelid is connected to an upper end of the lower eyelid, the gap may be formed between the upper eyelid and the lower eyelid, and the opening detection unit 111 may detect the opening degree of the eye through the gap between the upper eyelid and the lower eyelid. Further, the opening detection unit 111 may detect the upper eyelid and the lower eyelid in 3 dimensions through a convolutional neural network (CNN), and the CNN may be constituted by at least one convolutional layer, a pooling layer, and fully connected layers.

The opening degree of the eye may be divided into a closed state, a semi opened state, and a wide opened state. Here, the closed state means a state in which the pupil of the eyeball is fully covered by the upper eyelid and the lower eyelid and does not visibility, the semi opened state means a state in which the pupil of the eyeball is partially covered by the upper eyelid and the lower eyelid and is not exposed circularly, but has the visibility, and the wide opened state means a state in which the pupil of the eyeball is not covered by the upper eyelid and the lower eyelid and is circularly exposed and has the visibility.

Meanwhile, the opening detection unit 111 may detect makeup portions in the upper eyelid and the lower eyelid of the eye. Here, the makeup portion means a portion when the makeup (e.g., the eyelines, the eyelashes, tattoos, etc.) is processed in the upper eyelid and the lower eyelid. The opening detection unit 111 may detect the makeup portion through ResNet in the conventional neural network.

The pupil detection unit 112 may detect the pupil while the opening detection unit 111 detects the gap between the upper eyelid and the lower eyelid. Here, in the eyeball between the upper eyelid and the lower eyelid, the pupil shows a black color unlike another portion of the eyeball showing a white color, and as a result, the pupil detection unit 112 may detect a specific pupil between the upper eyelid and the lower eyelid.

Further, when the opening detection unit 111 already detects the makeup portion, the pupil detection unit 112 distinguishes and detects the pupil from the makeup portion to stably detect the pupil.

The operating unit 113 may generate the pupil coordinate based on the pupil detected by the pupil detection unit 112, and calculate the coordinate value for one point of the pupil. Here, the operating unit 113 may generate the pupil coordinate from the detected pupil by using the regression tree ensemble. Further, the calculated coordinate value of one point (e.g., a center) of the pupil may be used for checking the movement of the pupil.

The processing unit 114 is connected to the opening detection unit 111, the pupil detection unit 112, and the operating unit 113 to generate an image for the upper eyelid and the lower eyelid detected by the opening detection unit 111, and the pupil detected by the pupil detection unit 112, and the coordinate value of one point of the pupil calculated by the operating unit 113 may be included in the image.

Further, the processing unit 114 may transmit or receive a signal. For example, the processing unit 114 may generate a signal (e.g., an image signal according to the image) and transmit the generated signal to the control unit 102, and receive the signal from the control unit 102.

Meanwhile, the detection unit 101 according to the embodiment may further include a control command output unit 115. The control command output unit 115 may output a command voice. Here, the command voice means a voice including a command (e.g., ‘look right’, ‘look left’, etc.) that induces the movement of pupil to the user. The user may move the pupil by moving the eyeball in response to the command voice output from the control command output unit 115. Here, the pupil detection unit 112 may detect the moving pupil, and the processing unit 114 may generate an image according to the movement of the pupil and make the output command voice be included in the image. Further, the control command output unit 115 may output a command screen. Here, the command screen may mean a text including the command (e.g., ‘look right’, ‘look left’. etc.) that induces the movement of pupil to the user.

The control unit 102 may be connected to the detection unit 101, may receive the image signal according to the image from the detection unit 101, and store the image according to the image signal. Further, the control unit 102 may generate the signal and transmit the generated signal to the expert terminal 103 and the guardian terminal 104 in addition to the detection unit 101, and receive the signal from the expert terminal 103 and the guardian terminal 104.

Meanwhile, when the image includes a coordinate value of one point of the pupil, which is a set value or more or includes a coordinate value of one point of the pupil changed at a set period or more while the control unit 102 receives the image signal according to the image and stores the image according to the image signal, the control unit 102 may transmit the image signal according to the stored image to the expert terminal 103 or the guardian terminal 104. Here, the set value of the coordinate value of one point of the pupil and the set period of the coordinate value of one point of the pupil mean threshold numerical values for the coordinate value of one point of the pupil according to the movement of the pupil when the user is in the abnormal health state. The image according to the image signal transmitted from the control unit 102 may be output to the expert terminal 103 or the guardian terminal 104.

Further, when the image includes the command voice and a coordinate value of one point of the pupil, which changed at a velocity of a set value or less while the control unit 102 receives the image signal according to the image and stores the image according to the image signal, the control unit 102 may transmit the image signal according to the stored image to the expert terminal 103 or the guardian terminal 104. Here, the set value of the coordinate value of one point of the pupil mean a threshold numerical value for the coordinate value of one point of the pupil according to the movement of the pupil when the user is in an abnormal health state because the movement of the pupil of the user according to the command voice is not smooth. The image according to the image signal transmitted from the control unit 102 may be output to the expert terminal 103 or the guardian terminal 104.

The expert terminal 103 is connected to the control unit 102 to receive the signal from the control unit 102. Here, the when the signal is the image signal according to the image, the expert terminal 103 may output the image according to the image signal. When at least one of the coordinate value of one point of the pupil, the command voice, and the command screen are included in the image, the expert terminal 103 may output at least one of the coordinate value of one point of the pupil, the command voice, and the command screen jointly with the image.

An expert may check the movement of the pupil of the user through the output image, and diagnose the health state of the user. Here, the expert may be preferably a medical worker such as a doctor capable of diagnosing the health state of the user through the movement of the pupil.

Further, the expert terminal 103 may be manipulated by the expert and input with a comment. Here, the comment means statement-type contents according to the health state of the user diagnosed by the expert, and may be, for example, ‘The user is currently healthy.’, ‘The abnormal health of the user is detected. The user should be accurately diagnosed by visiting a hospital.’, ‘The user should be quickly hospitalized and treated.’, etc. In addition, a comment signal according to the comment may be generated from the expert terminal 103 and the comment signal may be transmitted to the control unit 102. Here, the control unit 102 may store the comment signal in response to the image.

Further, the expert terminal 103 may be manipulated by the expert and input with a command for the movement of the pupil. Here, the command signal for the movement of the pupil according to the command for the movement of the pupil may be generated from the expert terminal 103 and the command signal for the movement of the pupil may be transmitted to the control unit 102. Here, the control unit 102 may transmit the command signal for the movement of the pupil to the detection unit 101. In addition, the detection unit 101 may receive the command signal of the movement of the pupil from the control unit 102, and output a voice or a screen corresponding to the command signal for the movement of the pupil. In addition, the detection unit 101 may output the command signal in order to induce the movement of the pupil of the user. Here, the command signal for the movement of the pupil may include a command voice and a command screen for inducing the movement of the pupil of the user.

Further, the expert terminal 103 may include an expert interface for being input with the command for the movement of the pupil. The expert interface may be a portion output so that the expert conveniently inputs the command into to a screen of the expert terminal 103.

Here, the expert interface may include a screen mark movement layer for the expert to move a mark, a voice input layer for the expert to input the voice, and a specific behavior induction layer for the expert to indicate an object that induces a specific behavior.

For example, the expert drag the mark displayed on the screen of the expert terminal 103 and moves the mark to the screen mark movement layer to make only a required mark to remain in the screen.

As another example, when the expert touches the voice input layer displayed on the screen of the expert terminal 103, a voice spoken by the expert is recorded while a microphone of the expert terminal 103 is turned on and when the expert touches the voice input layer again, the recording may be completed.

As yet another example, when the expert touches the specific behavior induction layer displayed on the screen of the expert terminal 103, an arrow and a content input window through which may be selected by the expert are shown and the expert may input a specific-direction arrow and a text indicating a specific behavior.

Specific contents regarding the expert interface described above are just an example, and the present invention is not limited thereto.

The guardian terminal 104 is connected to the control unit 102 to receive the signal from the control unit 102. Here, when the signal is the image signal according to the image, the guardian terminal 104 may output the image according to the image signal. When the coordinate value of one point of the pupil or the command voice is included in the image, the guardian terminal 104 may output the coordinate value of one point of the pupil or the command voice jointly with the image.

A guardian may check the movement of the pupil of the user through the output image, and check the health state of the user. Here, when the user is a minor, the guardian may be a parent or a guardian of the user, and when the user is an adult, the guardian may be a spouse of the user, a child which is an adult, or a guardian.

Further, the guardian terminal 104 may receive the comment signal according to the comment from the control unit 102. Here, the comment by the expert may be output to the guardian terminal 104. The guardian may check the comment of the expert for the health state of the user through the guardian terminal 104 and take an action for the user according to the comment.

Meanwhile, the expert terminal 103 or the guardian terminal 104 may periodically receive the image signal according to the image stored in the control unit 102 from the control unit 102. As a result, the expert terminal 103 or the guardian terminal 104 may confirm the movement of the pupil of the user periodically through the image, and confirm the health state of the user.

In the embodiment, the expert terminal 103 or the guardian terminal 104 may be implemented by a computer which may access a remote server or terminal through the network. Here, the computer may include, for example, a notebook, a desktop, a laptop, etc., installed with a WEB Browser. Further, the expert terminal 103 and the guardian terminal 104 may be implemented by a terminal device which may access the remote server or terminal through the network 10. The terminal device which is, for example, a wireless communication device with guaranteed portability and mobility may include all types of handheld based wireless communication devices including Personal Communication System (PCS), Global System for Mobile Communications (GSM), Personal Digital Cellular (PDC), Personal Handyphone System (PHS), Personal Digital Assistant (PDA), International Mobile Telecommunication (IMT)-2000, Code Division Multiple Access (CDMA)-2000, W-Code Division Multiple Access (W-CDMA), Wireless Broadband Internet (Wibro) terminals, smartphones, smartpads, tablet PCs, etc.

The detection unit 101 in the medical examination system 100 according to the embodiment may detect makeup-processed portions of the upper eyelid and the lower eyelid of the eye in the semi opened state and the wide opened state of the eye, and detect the pupil by distinguishing from the makeup-processed portion. As a result, even when the pupil is partially covered by the eyelid and the makeup processing for the eye is achieved, the pupil is stably detected to achieve tracking according to the movement. That is, in order to detect and track the pupil, a request for widely opening the eye or removing the makeup may not be made if the eye is not closed. Therefore, the medical examination system 100 according to the embodiment easily detects the pupil of a person (e.g., Asian) having a relatively small eye to easily achieve the tracking of the pupil.

Further, the detection unit 101 in the medical examination system 100 according to the embodiment may generate an image including the detected upper eyelid, lower eyelid, and pupil, and make the coordinate value of one point of the pupil and the command signal be included in the image. Further, the control unit 102 may receive the image signal according to the image from the detection unit 101 and store the image corresponding to the image signal.

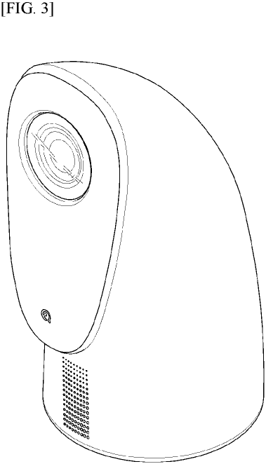

The control unit 102 may generate the image signal according to the image and transmit the generated image signal to the expert terminal 103 or the guardian terminal 104. The expert and the guardian may confirm the pupil movement of the user through the images according to the image signals output through the expert terminal 103 or the guardian terminal 104, respectively. In particular, the expert may confirm the health state of the user based on the pupil movement of the user through the expert terminal 103, and input the comment into the expert terminal 103. Here, the control unit 102 may receive the comment signal according to the comment of the expert from the expert terminal 103, and transmit the comment signal to the guardian terminal 104. The guardian may receive the comment signal through the guardian terminal 104 and confirm the comment of the expert for the comment signal, and take an action for the user according to the comment of the expert. Accordingly, the medical examination system 100 according to the embodiment may detect and track the pupil of the user by using the detection unit 101 which is the form of the camera, and may be diagnosed by the expert through the expert terminal 103 which is the form of the terminal positioned remotely to diagnose the health state of the user even at low cost and take an appropriate action for the user according to the diagnosis of the expert. The detection unit 101 may be implemented by various types of devices, and when the detection unit 101 illustrated in FIG. 3 is placed on a desk, it may become possible to easily examine the pupil in an everyday life.

Meanwhile, the control unit 102 in the medical examination system 100 according to the embodiment is connected to a plurality of detection units 101 to receive image signal according to images from the detection units 101, respectively. Here, the control unit 102 may detect health states of users corresponding to the detection units 101, respectively based on the movement of the pupil through the coordinate value of one point of the pupil included in the image, and classify the users according to the detected health states. In particular, the control unit 102 may set a transmission order of the image signals according to the images of the users according to the health states of the users. Here, the control unit 102 may set the image signal according the image of the user which is in a relatively dangerous health state as a priority and preferentially transmit the image signal to the expert terminal 103.

For example, the control unit 102 may classify the health states of the users into a normal group, a suspicious group, a risk group, and a high risk group. Here, the control unit 102 may set an order according to a risk level among the users included in the high risk group, and transmits the image signal according to the image corresponding to the user to the expert terminal 103 according to the order. As a result, the expert may diagnose the health state by preferentially checking the image corresponding to the user which is in the relatively dangerous health state through the expert terminal 103, and the user may be preferentially diagnosed and treated in the relatively dangerous health state.

Further, the guardian may input user information and acquaintance information in advance through the guardian terminal 104, and transmit and store the user information and the acquaintance information to and in the control unit 102. Here, the user information may be a name and an address of the user (e.g., an address space in which the detection unit 101 is installed) and the acquaintance information may be a name and a cellular phone number of an acquaintance (e.g., a person who lives near the user, and may knows the user or help the user). The control unit 102 may continuously track a location of the terminal of the acquaintance and track the location of the acquaintance based on the acquaintance information.

When the control unit 102 receives the image signal according to the image corresponding to the user who in the dangerous health state (e.g., the high risk group), the control unit 102 may generate a notification signal according to a notification message, and transmit the generated notification signal to the terminal of the acquaintance closest to the user according to prestored user information. The acquaintance closest to the user may receive the notification signal through the terminal thereof, and confirm the notification message. Here, the notification message may include a behavior matter for the acquaintance who confirms the health state of a current user and the notification message, and may be, for example, "Mr./Ms. ΔΔΔ, the user who lives at □□ is in a dangerous state, so please, help the user.". Further, the notification message may include a response menu, and when the acquaintance touches the response menu, a response signal may be generated in the terminal of the acquaintance and transmitted to the control unit 102. Here, the acquaintance preferably touches the response menu in a state of being capable of act according to the notification message.

Meanwhile, after the notification signal is transmitted to the terminal of the acquaintance, when the control unit 102 does not receive the response signal within a predetermined time (e.g., 10 seconds, 30 seconds, 1 minute, etc.), the control unit 102 may transmit the notification signal according to the notification message to the terminal of the acquaintance closer to the user in a next order. For example, the notification message may be "Mr./Ms. ooo, the user who lives at □□ is in a dangerous state, but there is a situation in which it is difficult for ΔΔΔ to help the user. Therefore, please, help the user.". The notification message may be displayed in the terminal of the acquaintance closer to the user in the next order differently from the terminal of the acquaintance closest to the user.

Further, the control unit 102 may transmit acquaintance information and location information of the acquaintance who transmits the response signal to the guardian terminal 104. Here, the guardian may confirm the acquaintance information and the location of the acquaintance who moves toward the user through the guardian terminal 104.

In addition, when a distance between the location of the acquaintance who touches the response menu included in the notification message and the location of the user is a set value or more, the control unit 102 selects the location of the acquaintance who touches the response menu as a departure and selects the address of the user as a destination to generate a call signal for calling a sharing vehicle such as a taxi, etc. Further, the control unit 102 may transmit vehicle information (e.g., a current vehicle location, a departure arrival time, a vehicle number, etc.) for the sharing vehicle which responds to the call signal to the terminal of the acquaintance who touches the response menu. As a result, the acquaintance may use the provided vehicle and in some cases, may cancel the call signal.

As described above, the control unit 102 may transmit a notification message according to the health state of the user, in particular, the dangerous health state to the terminal of the acquaintance according to the order of the acquaintance close to the user based on the acquaintance information input by the guardian terminal 104 in advance and prestored. Accordingly, the medical examination system 100 according to the embodiment may provide an actual help from the acquaintance to the user according to the health state.

Further, the control unit 102 may receive and store location information of a plurality of emergency rescue centers in advance. When the control unit 102 receives the image signal according to the image corresponding to the user who in the dangerous health state (e.g., the high risk group), the control unit 102 may transmit an emergency signal according to an emergency message to an emergency rescue center closest to the user according to the user information of the user in the prestored location information of the emergency rescue centers. The emergency rescue center may confirm the emergency message through the terminal. Here, the emergency message may include the health state and address of the current user. As a result, the emergency rescue center may provide an emergency crew and an ambulance for the user.

Although the technical idea of the present invention is described in detail with a preferred embodiment, the technical idea of the present invention is not limited to the above embodiments, and various modifications and changes can be made by those skilled in the art within the scope of the technical idea of the present invention.

### Mode for Invention

Related contents in the best mode for carrying out the present invention are described as above.

### Industrial Applicability

The present invention can be used in a medical examination apparatus, a medical examination system, etc.

1. A method for tracking a pupil, the method comprising:
detecting an upper eyelid and a lower eyelid in eyes;
detecting an opening degree of the eye;
detecting the pupil between the upper eyelid and the lower eyelid;
generating a pupil coordinate in accordance with the detected pupil; and
calculating a coordinate value for one point of the pupil in the pupil coordinate.

2. The method for tracking a pupil of claim 1, wherein the detecting of the upper eyelid and the lower eyelid in the eyes includes
detecting a makeup portion between the upper eyelid and the lower eyelid, and
is achieved by a convolutional neural network.

3. The method for tracking a pupil of claim 1, wherein in the detecting of the pupil between the upper eyelid and the lower eyelid,
in the opening degree of the eye, the pupil is detected between the upper eyelid and the lower eyelid while the upper eyelid and the lower eyelid are spaced apart from each other so that the pupil has a visibility.

4. The method for tracking a pupil of claim 3, wherein in the detecting of the pupil between the upper eyelid and the lower eyelid,
pupil tracking technologies are differently applied according to the opening degree of the eye.

5. A medical examination system comprising:
a detection unit detecting an eye of a user, detecting an opening degree of the eye and a pupil through the opened eye, and generating an image of the detected eye;
a control unit connected to the detection unit, and receiving an image signal according to the image from the detection unit and storing the image according to the image signal; and
an expert terminal connected to the control unit to receive the image signal according to the image from the control unit, and output the image according to the image signal and receive a comment,
wherein the detection unit detects an upper eyelid and a lower eyelid in the eye, detects the opening degree of the eye, and detects the pupil between the upper eyelid and the lower eyelid, and
the expert terminal receives a comment through an expert interface and transmits a comment signal according to the comment to the control unit.

6. The medical examination system of claim 5, wherein the detecting unit includes
an opening detection unit detecting the eye of the user, detecting the upper eyelid and the lower eyelid of the eye, and detecting the opening degree of the eye,
a pupil detection unit detecting the pupil between the upper eyelid and the lower eyelid detected by the opening detection unit,
an operating unit generating a pupil coordinate according to the pupil detected by the pupil detection unit, and calculating a coordinate according to one point of the pupil, and
a processing unit connected to the opening detection unit, the pupil detection unit, and the operating unit to generate images for the upper eyelid and the lower eyelid detected by the opening detection unit, and the pupil detected by the pupil detection unit, include a coordinate value of one point of the pupil calculated by the operating unit be in the image, and transmit and receiving a signal.

7. The medical examination system of claim 6, wherein the processing unit generates the image signal according to the image and transmits the image signal to the control unit,
the control unit receives an image signal according to an image including a movement of the pupil corresponding to a case where the user is in an abnormal health state and transmits the image signal to the expert terminal, and
the expert terminal outputs the image according to the transmitted image signal, and receives a comment according to the output image, and generates the comment signal according to the comment and transmits the generated comment signal to the control unit.

8. The medical examination system of claim 7, further comprising:
a guardian terminal connected to the control unit to receive the signal from the control unit,
wherein the control unit receives an image signal according to the image including the movement of the pupil corresponding to the case where the user is in the abnormal health state and transmits the image signal to the guardian terminal, and transmits the comment signal according to the comment transmitted from the expert terminal to the guardian terminal, and
the guardian terminal outputs the image according to the transmitted image signal and the comment according to the comment signal.

9. The medical examination system of claim 6, wherein the detection unit further includes a control command output unit capable of outputting a command voice and a command screen, and
the control command output unit outputs the command voice and the command screen to induce the movement of the pupil of the user.

10. The medical examination system of claim 6, wherein the opening detection unit detects a makeup portion between the upper eyelid and the lower eyelid.

11. The medical examination system of claim 6, wherein when the opening detection unit detects the opening degree of the eye in which the upper eyelid and the lower eyelid are spaced apart from each other so that the pupil has a visibility, the pupil detection unit detects the pupil between the upper eyelid and the lower eyelid.

12. The medical examination system of claim 5, wherein the expert terminal receives a command of the movement of the pupil through the expert interface and transmits the command signal of the movement of the pupil to the control unit.

13. The medical examination system of claim 12, wherein the detection unit receives the command signal of the movement of the pupil from the control unit, and outputs the command signal to induce the movement of the pupil of the user.

14. The medical examination system of claim 12, wherein the expert terminal includes the expert interface for receiving the command of the movement of the pupil, and
the expert interface includes
a screen mark movement layer capable of moving a mark by an expert,
a voice input layer through which the expert is capable of inputting a voice, and
a specific behavior induction layer through which the expert is capable of displaying an object for inducing a specific behavior.
